(19) United States
(12) Patent Application Publication
(10) Pub. No.: US 2016/0115528 A1
(43) Pub. Date: Apr. 28, 2016

(54) MEASURING DEVICE AND SYSTEM FOR PERFORMING MELTING CURVE ANALYSIS OF A DNA MICROARRAY AND UTILIZATION OF A FLUORESCENCE DETECTOR ARRAY FOR ANALYSIS

(71) Applicant: Fraunhofer-Gesellschaft zur Foerderung der angewandten Forschung e.V., Munich (DE)

(72) Inventors: Anna OHLANDER, Munich (DE); Thomas GANKA, Munich (DE); Karlheinz BOCK, Alling (DE)

(21) Appl. No.: 14/921,275

(22) Filed: Oct. 23, 2015

(30) Foreign Application Priority Data
Oct. 24, 2014 (DE) 102014221734.2

(51) Int. Cl. C12Q 1/68; G01N 25/04; G01N 21/64

(52) U.S. Cl. CPC C12Q 1/6818; G01N 21/6454; G01N 21/6428; G01N 25/04; G01N 2021/6432

(57) ABSTRACT

What is shown is a measuring device for simultaneously analyzing melting curves of DNA samples in DNA microarrays with the aid of a fluorescence detector array. Embodiments show monitoring of melting curves of DNA microarrays (dynamic fluorescence measurement) by means of silicon photomultipliers (SiPM) or other photodetectors such as, e.g., PIN diodes (positive intrinsic negative diodes), avalanche photodiodes (APD), or photomultiplier tubes (PMT). The DNA microarray is applied, along with a thin-film heating element, to a substrate made of plastic or glass or is integrated in a microfluidic channel.

### CROSS-REFERENCE TO RELATED APPLICATIONS

This application claims priority from German Patent Application No. 10 2014 221 734.2, which was filed on Oct. 24, 2014, and is incorporated herein in its entirety by reference.

The present invention relates to a measuring device and a system for performing melting curve analysis of at least one DNA sample with the aid of a fluorescence detector array. Embodiments show monitoring of melting curves of DNA microarrays (dynamic fluorescence measurement) by means of silicon photomultipliers (SiPM) or other photodetectors such as, e.g., PIN diodes (positive intrinsic negative diodes), avalanche photodiodes (APD), or photomultiplier tubes (PMT).

### BACKGROUND OF THE INVENTION

Molecular diagnostics will play an important part in future global health as it enables early diagnosis with high accuracy, disease monitoring and risk detection as well as customizing of medication for each individual patient. Melting curve analysis (MCA) is a common method of analyzing variations in DNA samples, in particular of DNA sequences (deoxyribonucleic acid). Said method is used in a multitude of applications such as typing of disease and cancer loci, biomarker discovery, typing of transgenic plants and animals, pathogen detection and genotyping and methylation studies.

The method is based on exposing double-stranded DNA duplexes to a temperature gradient and monitor the decaying fluorescence intensity that results from the double stranded DNA breaking up into single-stranded DNA due to the increase in temperature. The method is sensitive enough to distinguish between sequence variances down to a single base, which makes it an efficient tool in detecting single base mutations or so-called single nucleotide polymorphisms (SNPs). SNPs are the most common genetic variation between individuals and can be used as diagnostic markers for various medical conditions. Conventional MCA is performed in a liquid state using standard PCR (polymerase chain reaction) equipment (e.g., by Roche, Qiagen, Applied Biosystems).

Brookes et al. introduced surface bound MCA under the name DASH (dynamic allele-specific hybridization) based on a microtiter plate format and a cellulose membrane for enhanced multiplexing of assays or analyses [1], [2].

As molecular diagnostic methods rely on labor-intense assay protocols in a centralized laboratory which involve high costs and long waiting times, efforts to automatize the assays and detach the technology from centralized laboratories by using lab-on-a-chip and point-of-care concepts represent a growing field of research. In order to enable a field-deployable system, these systems depend on components that are compact, low in cost, and low power consuming without comprising performance.

Fluorescence detection is one of the most common detection principles in lab-on-a-chip devices. One of the aims of these systems is to reduce reaction volumes. Common fluorescence detectors are photomultiplier tubes (PMTs) and avalanche photodiodes (APDs) which can detect single photons at high speed but do not provide spatial resolution. PMTs further have the disadvantage of being bulky and expensive and of requiring high operation voltages. Charge coupled devices (CCDs) provide spatial resolution but at the cost of reduced sensitivity and long integration times resulting therefrom.

Recently, MCA (melting curve analysis) on DNA microarrays was demonstrated with a microfluidic lab-on-a-chip system with integrated thin-film microheaters for detecting SNPs (single nucleotide polymorphisms) [3], [4], [5], [6]. In order to determine the intensity of fluorescence of the individual DNA samples, microscope images (CCD camera) with subsequent image analysis were used, which prevented the analysis from being applied or performed in a portable format.

### SUMMARY

According to an embodiment, a measuring device for performing melting curve analysis of at least one DNA sample may have: a two-dimensional DNA microarray for accommodating the at least one DNA sample; a two-dimensional fluorescence detector array including at least one fluorescence detector; and an integrated heating element for heating the DNA sample applied to the DNA microarray.

According to another embodiment, a system may have: a measuring device as claimed in claim 1; and an excitation light source which excites the at least one DNA sample to fluoresce.

Another embodiment may include utilization of a fluorescence detector array for performing melting curve analysis of a DNA sample, at least one fluorescence detector of the fluorescence detector array being a silicon photomultiplier (SiPM).

According to another embodiment, a measuring device for performing melting curve analysis of Y×Z DNA samples may have: a DNA microarray for accommodating the Y DNA samples arranged along a Y extension and the Z DNA samples arranged along a Z extension; a fluorescence detector array including Y×Z separate fluorescence detectors, Y fluorescence detectors being arranged along a Y extension and Z fluorescence detectors being arranged along a Z extension, or a monolithic Y×Z multichannel fluorescence detector, Y channels of the multichannel fluorescence detector being arranged along a Y extension and Z channels of the multichannel fluorescence detector being arranged along a Z extension; and an integrated heating element for heating the DNA samples applied to the DNA microarray; the measuring device being configured to analyse the Y×Z DNA samples on the DNA microarray at the same time; and each of the Y×Z DNA samples being aligned to be spatially opposite one of the Y×Z fluorescence detectors of the fluorescence detector array or one of the Y×Z channels des multichannel fluorescence detector and thus being associated with a corresponding fluorescence detector or channel.

Another embodiment may include of a fluorescence detector array for performing melting curve analysis of a DNA sample, at least one fluorescence detector of the fluorescence detector array including a group of individual silicon photomultipliers (SiPM).

Embodiments show a measuring device for performing melting curve analysis of at least one DNA sample. The measuring device comprises a DNA microarray for accommodating the at least one DNA sample and a fluorescence detector array comprising at least one fluorescence detector. In addition, the measuring device includes an integrated heating element for heating the DNA sample applied to the DNA microarray.

The invention is based on the finding that in order to achieve a melting curve having a high temporal resolution and to achieve a fast temperature rise, a low heat capacity and a geometric proximity of heater and sample are advantageous. To this end, an integrated thin-film heater is useful. Unlike external sources of heat (such as Peltier heating elements, for example), such heaters have been shown not to cause the sample to migrate out of the focus of the microscope due to thermal expansion and, therefore, to enable reliable measurement of fluorescence. Further advantages consist in that the melting curve analysis device as an integrated unit may now be built in a very compact manner.

In accordance with further embodiments, utilization of highly sensitive optical detectors such as silicon photomultipliers, for example, advantageously contributes to the compact design. Therefore, embodiments show the fluorescence detector which comprises a silicon photomultiplier, an avalanche photodiode, or a PIN (positive intrinsic negative) diode. This is advantageous since, e.g., the silicon photomultiplier exhibits an amplification for weak optical signals in the range from 500,000 to 1,000,000. Utilization of a fluorescence detector exhibiting a high level of intrinsic amplification of the optical input signals enables examining relatively small DNA samples, which overall results in a relatively small design of the measuring device. The background to this is that a relatively small DNA sample exhibits comparatively less fluorescence than a relatively large biochemical sample which, however, may be compensated for by a high level of intrinsic amplification of the fluorescence detector, which transforms even a small level of input light intensity to a measureable output signal.

In accordance with embodiments, the power of the input light intensity may exist in the form of few photons per second from which an output current is generated which corresponds to the product of the number of photons and the amplification of the photodetector (e.g., 5*10̂5). For example, if the input light intensity is 100 photons per second, this will result in an output current of the fluorescence detector which is generated by at least 5*10̂7 electrons per second.

Further embodiments show that the fluorescence detector is configured to react with an amplified output signal to a power of the optical input signal that results from the fluorescence of at least one DNA sample.

In addition, the fluorescence detector may be configured to detect a change in the fluorescence intensity of the at least one DNA sample that is caused by a change in a nucleic-acid structure that is due to a temperature rise. A change in the fluorescence may occur when the DNA sample is heated, for example when double-stranded DNA is transformed to single-stranded DNA.

In accordance with a further embodiment, the measuring device is a fluorescence detector array comprising a plurality of fluorescence detectors or a monolithic multichannel fluorescence detector. Each of the plurality of fluorescence detectors or each channel of the multichannel fluorescence detector may be coupled to a DNA sample by means of an optical waveguide or imaging optics. Each of the DNA samples is thus associated with a corresponding fluorescence detector. Said association may also be performed by aligning the DNA sample to be spatially directly opposite the fluorescence detector. Moreover, the measuring device may be configured to simultaneously analyze a plurality of DNA samples on the microarray. To this end, each of the plurality of DNA samples may be associated with a fluorescence detector of the fluorescence detector array. Furthermore, the measuring device may comprise a surface area smaller than 3 mm² or smaller than 10 mm² or smaller than 25 mm². Embodiments enable for the measuring device to be portable.

Further embodiments show that excitation of the fluorescence takes place when a direct-light method is used with a transparent substrate and a transparent or semi-transparent integrated heating element. A different embodiment shows that excitation of the fluorescence takes place when an incident-light method is used. The substrate may consist of a die-cast plastic part, a plastic film, or a glass carrier. For evaluation using transmission, or a direct-light method, the substrate is to be configured to be transparent or semi-transparent; when reflection, or an incident-light method, is used, it may also be opaque.

Embodiments show a system comprising a measuring device and an excitation light source which excites the DNA sample to fluoresce.

In addition, the fluorescence detector array for performing a melting curve analysis of the DNA sample may be used with at least one fluorescence detector, at least one fluorescence detector of the fluorescence detector array being a silicon photomultiplier (SiPM).

### BRIEF DESCRIPTION OF THE DRAWINGS

Embodiments of the present invention will be detailed subsequently referring to the appended drawings, in which:

FIG. 1a shows a schematic top view of a measuring device for performing a melting curve analysis of at least one DNA sample;

FIG. 1b shows a schematic side view of the measuring device shown in FIG. 1a;

FIG. 1c shows the measuring device shown in FIG. 1b, with a reduced degree of magnification;

FIG. 2a shows a schematic side view of an incident-light setup for performing melting curve analysis;

FIG. 2b shows a schematic side view of a direct-light setup for performing melting curve analysis;

FIG. 3a shows a schematic representation of a side view of the excitation light source, of a detector, and of a DNA sample;

FIG. 3b shows a schematic representation of a side view of the measurement setup of FIG. 3a, the difference being that the excitation light source and the detector are arranged on different sides of the DNA sample;

FIG. 4a shows exemplary melting curves for matched and mismatched DNA samples, recorded with two SiPMs and plotted as a current intensity, normalized to starting value 1 and final value 0, over the temperature;

FIG. 4b shows exemplary melting curves for the DNA samples shown in FIG. 4a, plotted as an intensity, normalized to starting value 1 and final value 0, over the temperature, that were determined by using a microscope CCD camera and by extracting the respective fluorescence intensity by means of an image software;

FIG. 5a shows exemplary melting curves wherein the DNA cleavage is additionally plotted over the temperature curve;

FIG. 5b shows the derivatives of the exemplary melting curves of FIG. 5a; and

FIG. 6a to 6d show a schematic representation of potential fluorescence marker systems within the visible spectral range between 400 and 800 nm.

### DETAILED DESCRIPTION OF THE INVENTION

In the following description of the figures, elements that are identical or have identical actions shall be provided with identical reference numerals, so that their descriptions are interchangeable in the various embodiments.

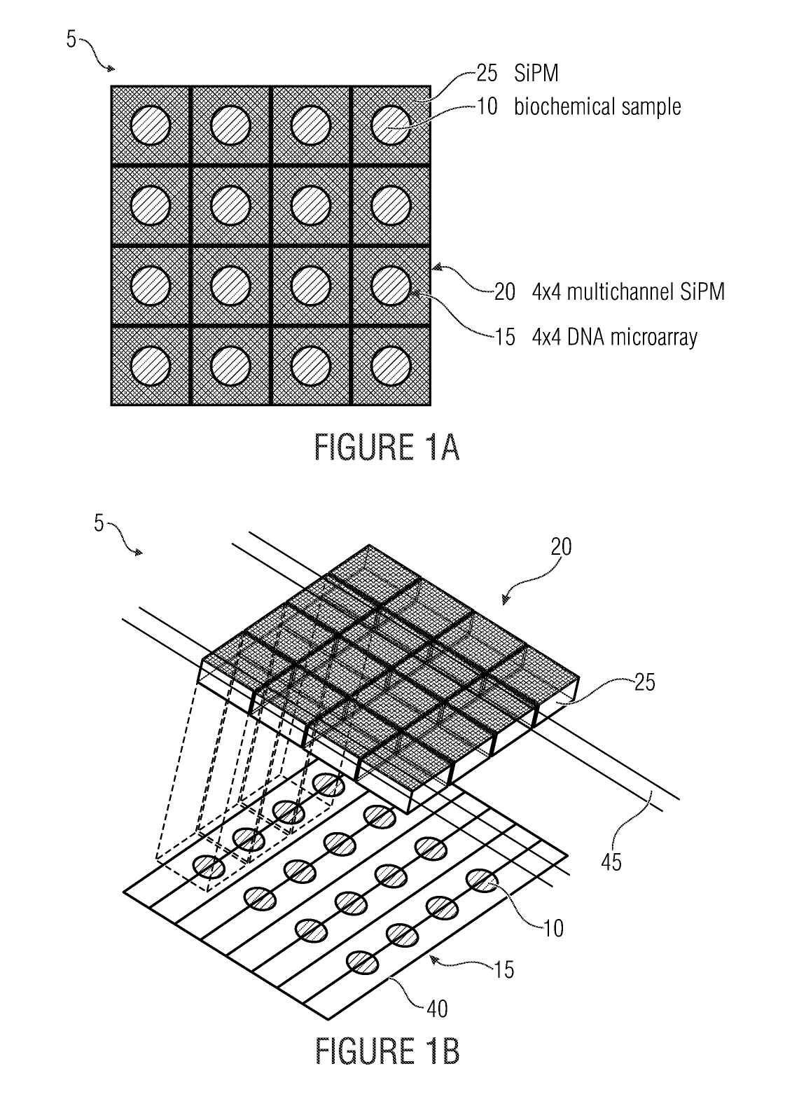

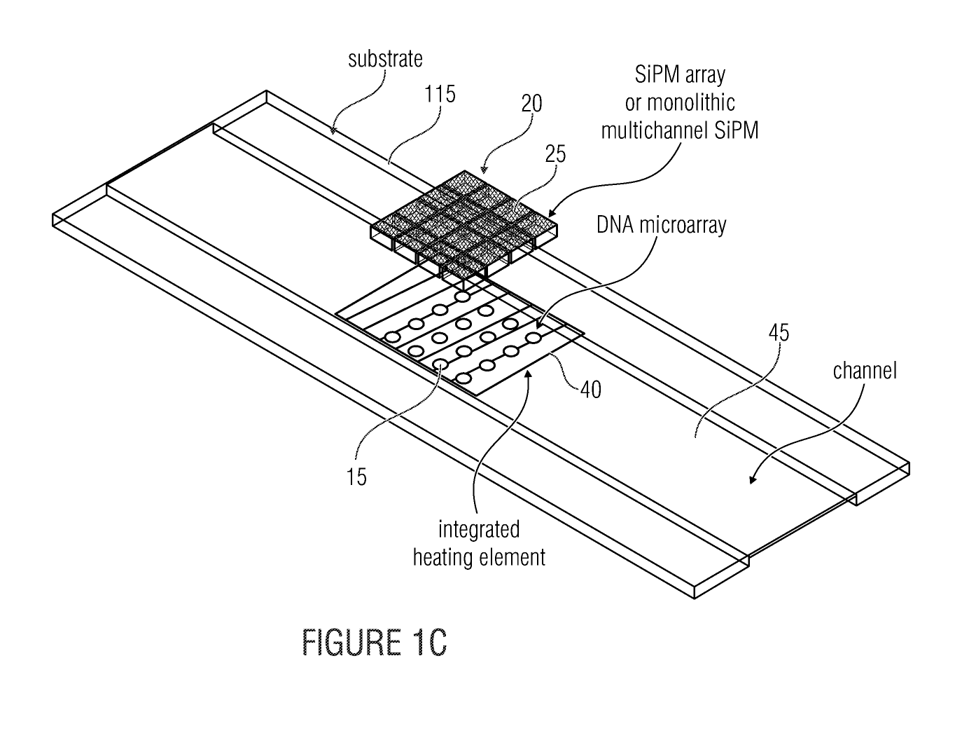

FIG. 1a shows a schematic top view of a measuring device 5 for analyzing at least one DNA sample 10 which comprises a microarray 15 for accommodating the at least one DNA sample 10 and a fluorescence detector array 20 comprising at least one fluorescence detector 25. Here, the fluorescence detector array 20, which is configured as a 4×4 array, is arranged above the microarray 15, which is also configured as a 4×4 array, so that each photodetector of the fluorescence detector array 20 can analyze a cell of the 4×4 microarray. In this view, an integrated heating element 40 is concealed but is shown in FIG. 1b.

With this aim in view, the microarray 15 is a regular arrangement of DNA samples 10 which are simultaneously examined under identical environmental conditions such as temperature, pH value and buffer solution.

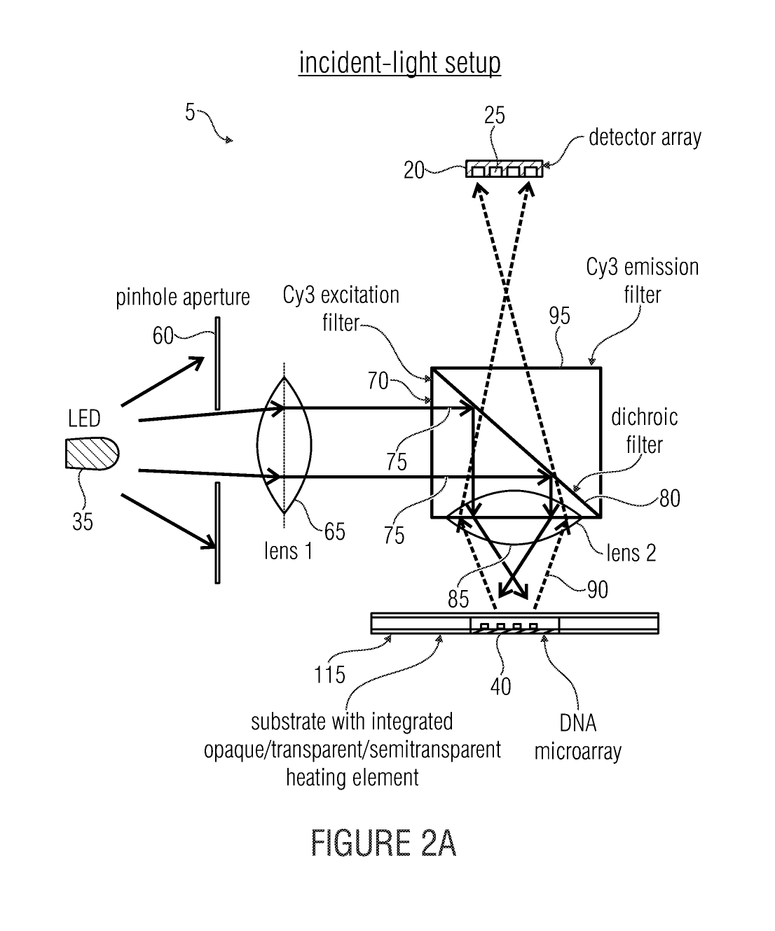

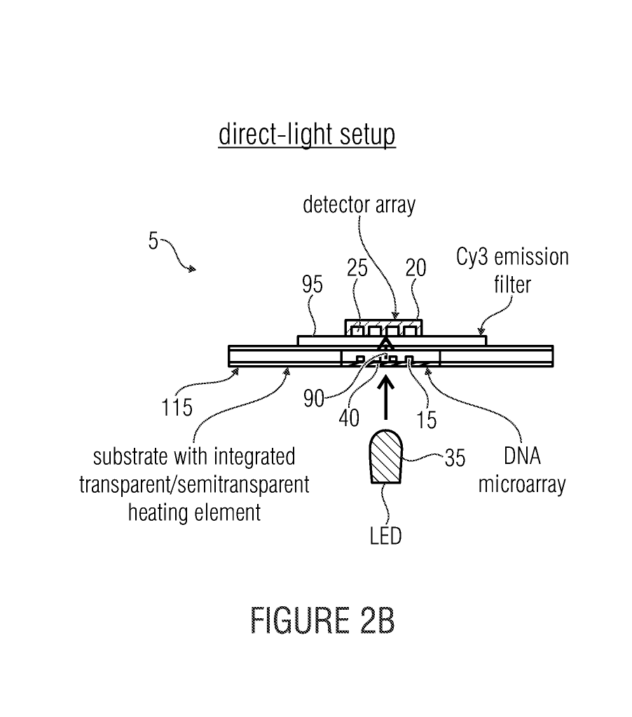

In fluorescence analysis, a DNA sample is illuminated with high-energy light of an excitation light source (e.g., UV light) and will fluoresce at a relatively long wavelength. The resulting, non-directional fluorescence radiation may be analyzed by using reflection or transmission in relation to the excitation light source. One will often select a geometric arrangement which enables separating the optical excitation path from the optical fluorescence path so as to achieve maximum suppression of background signals. The measurement processes will be explained in more detail with reference to FIGS. 2a and 2b.

For a melting curve that exhibits a high temporal resolution and for a rapid rise in temperature, a low heat capacity and geometric proximity of heater and sample are advantageous. To this end, an integrated thin-film heater 40 is useful. Thus, in the embodiments presented here, thermal expansion of the sample is prevented or at least minimized to such an extent that the sample remains within the focus of the microscope despite thermal expansion and enables reliable DNA analysis. Alternative utilization of external heat sources such as Peltier heating elements shows that due to thermal expansion, the sample migrates out of the focus of the microscope and renders reliable DNA analysis difficult or prevents it.

It shall be noted that in derogation from the arrangement shown in FIG. 1a which has 4×4 DNA samples 10 and/or fluorescence detectors 25, the DNA microarray 15 as well as the fluorescence detector array 20 may also include only one DNA sample 10 and/or one fluorescence detector 25 or, generally, a number of Y×Z DNA samples 10 and/or fluorescence detectors 25. If the measuring device includes a fluorescence detector array 20 having a plurality of fluorescence detectors 25 or one monolithic multichannel fluorescence detector 25, the measuring device may be configured to analyze a plurality of DNA samples 10 on the DNA microarray 15 at the same time.

Typically, analysis is performed such that the DNA sample 10 within the DNA microarray 15 is heated by means of the integrated heating element 40 (shown in FIGS. 1b and 2), during which process the fluorescence will characteristically change. The change in fluorescence is detected by means of the fluorescence detector 25 of the fluorescence detector array 20, it being possible to draw conclusions with regard to the sample by the very correlation between temperature and fluorescence. It has been shown that there is the need to examine samples 10 that are as small as possible (e.g., that have diameters of 10 to 100 μm), which, however, will also result in a smaller change in fluorescence. This is why it is advantageous to employ sensitive fluorescence detectors 25. In combination with the integrated heating element 40, they enable a small design which in addition to employment in a laboratory environment also offers the possibility of producing portable measuring devices 5 for melting curve analysis. To this end, the measuring device may be an integrated system built into a common housing.

In accordance with an embodiment, each of the plurality of DNA samples 10 may be associated with a fluorescence detector 25 of the fluorescence detector array 20, as is schematically indicated in FIG. 1b. FIG. 1b shows a schematic side view of the measuring device 5 shown in FIG. 1a. Association of the fluorescence detectors 25 with the DNA samples 10 is shown by the symbolic, dashed extension of the outer edges of the fluorescence detectors 25. FIG. 1c shows the device, which is shown in FIG. 1b, in a different section and/or a deviating level of magnification. In addition, FIGS. 1b and 1c show an optional, microfluidic channel 45 via which the DNA samples may be flushed away from the microarray, for example after an experiment has been performed. Likewise, this view shows the integrated heating element 40 manufactured, e.g., by means of a lab-on-a-chip process or a roll-to-roll process. The integrated heating element 40 is integrated within the DNA microarray, for example on a film, and is indirect heat contact with the DNA sample. The integrated heating element 40 may be set up as a micro-patterned thin-film heater, for example of a grid made of patterned thin-film copper. The grid includes an arrangement of narrow conductor lines having widths from 1 μm to 20 μm with interposed transparent regions. The thickness of such a thin-film heater ranges from 100 nm to 1 μm. This enables fabrication of a semi-transparent heater 40. In this context, the film substrate is transparent. For the embodiment of a direct-light fluorescence setup (cf. FIG. 2b), a transparent heater is advantageous.

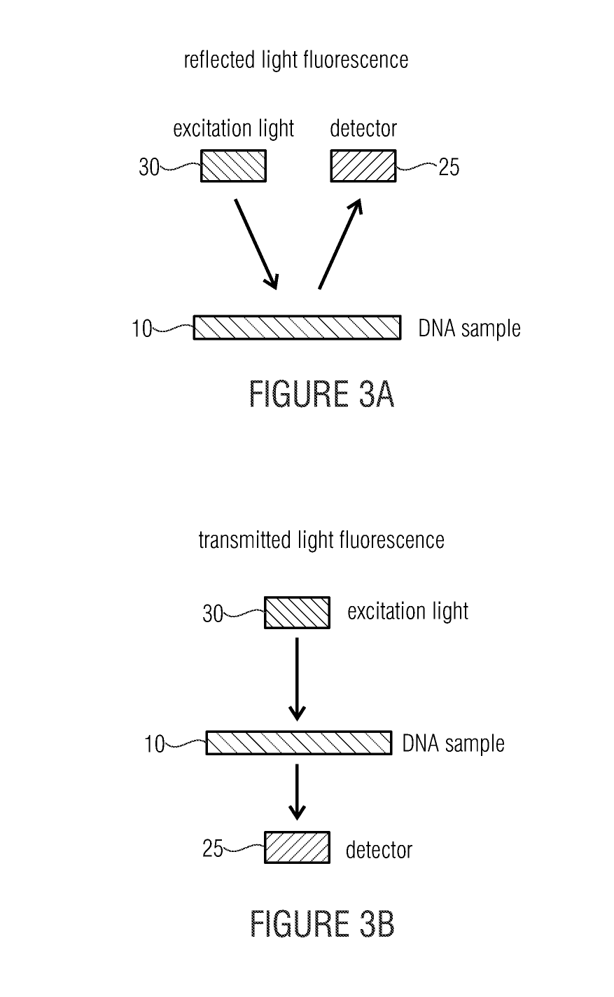

Alternatively, the photons of the DNA sample 10 that are emitted when said DNA sample fluoresces may be concentrated onto the pertinent fluorescence detector 25 by means of an optical waveguide or an optical system (cf. FIG. 2a and FIG. 3a). A plurality of optical waveguides forwards the fluorescence light from a sample 10 to the associated photomultiplier and/or the fluorescence detector 25. By means of imaging optics, the light of the samples arranged within the DNA microarray may be imaged onto the detector array. Utilization of the latter embodiment is advantageous in laboratories.

The described embodiment of incident-light fluorescence measurement is schematically depicted in FIG. 2a. An excitation light source 35 such as a green-colored LED, for example, transmits the excitation light through a pinhole 60, focusses it to a cyan 3 (Cy3) excitation filer 70 through a first lens 65. The excitation filter 70 limits the spectral range of the excitation light to a fixed wavelength, e.g., to 550 nm, and filters any further existing spectral components out of the former. The filtered excitation light 75 is deflected via a dichroic filter, or a dichroic mirror, 80 and is focused onto the DNA sample via a second lens 85. By the filtered excitation light 75, the DNA sample is excited to fluoresce and emits light 90 having a wavelength of 570 nm, for example. Said light is focused onto the fluorescence detector array 20 via the second lens 85 through the dichroic mirror 80, which is translucent to the wavelength of the light 90. Said focusing is performed such that the emitted light of a DNA sample is directed onto a corresponding fluorescence detector 25. Instead of a lens, an optical waveguide may also be used for this purpose, for example. Any undesired spectral components that do not stem from the fluorescence but are produced, for example, by background signals, are filtered out by the cyan 3 emission filter 95 (also referred to as a stop filter) arranged between the dichroic mirror 80 and the fluorescence detector array 20.

FIG. 2b shows the embodiment of the direct-light fluorescence setup. Association of the DNA samples 10 with a fluorescence detector 25 may here be advantageously achieved by arranging the detector array in direct proximity of the DNA sample array. In this context, each individual detector will receive only the fluorescence light of the DNA sample associated with it. To this end, the fluorescence markers are excited by using the incident-light method. Consequently, imaging optics or waveguides may be dispensed with. However, the cyan 3 filter is advantageous since, for example, the light of the excitation light source 35 should not impinge upon the frequency detector array so as to detect the fluorescence exclusively. Thus, a small design and an integrated device for melting curve analysis of DNA samples can be achieved.

In the embodiments described, the exciting light is blocked by a color filter arranged in front of the detector, and only the light emitted by the sample while it fluoresces is transmitted. In accordance with an embodiment, the fluorescence detector 25 may comprise a silicon photomultiplier for detecting fluorescence. Alternatively, the fluorescence detector 25 may also include a photodiode, e.g., an avalanche photodiode.

In accordance with embodiments, the measuring device 5 comprises, e.g., at a cross-sectional area of a lateral section through the fluorescence detector 25, an area of less than 3 mm². Embodiments show a cross-sectional area smaller than 2 mm², smaller than 1 mm², or smaller than 0.5 mm².

In an advantageous embodiment, a photodetector associated with a sample includes a group of many individual microavalanche photodiodes (SiPM). The signal of this group of microavalanche photodiodes is combined into an aggregate signal and is evaluated. Adjacent groups are electrically and optically separated from one another to avoid crosstalk between different channels. This offers the possibility of combining the high level of sensitivity, the low level of dark current, and the short response time of PMTs (photomultipliers) with the spatial resolution and the compact design of CCDs. Since the multichannel SiPMs can be produced from a single silicon component in a monolithic manner, they are very small, low in cost and exhibit low current consumption, which is ideal for portable point-of-care diagnostic systems.

By heating the at least one DNA sample 10, a change in the fluorescence intensity of said sample may be caused, which can be detected by the measuring device 5. The change in fluorescence intensity is due to a change in the nucleic-acid structure of the at least one DNA sample 10, which is why the measuring device is configured to detect the change in the nucleic-acid structure.

In detail, the fluorescence detector 25 is configured to react to an energy of an input light intensity, which is due to a fluorescence of the at least one DNA sample 10, with an amplified output signal. In accordance with an embodiment, the power of the optical input signal of the fluorescence detectors 25 is caused by at least one photon. The fluorescence detector array 20 transforms the energy of the photon to an electric charge pulse Q, which may include, on account of the intrinsic amplification G of the photodetector, the charge of several electrons. In the event of an avalanche photodiode, this intrinsic amplification is approximately 50 to 500. If, for example, 100 photons impinge upon the photodetector per second, an electrical current I=Q/t=G*e/t of 5,000 electrons per second is generated. The amplification of a silicon photomultiplier (e.g., G>5*10̂5) is sufficiently high to enable reaffirming the presence of individual photons. If Pin diodes are used, the fluorescence signal will be transformed to an electric measurement signal. As compared to, e.g., SiPM, APD, and PMT, this measurement signal is clearly smaller since Pin diodes exhibit no intrinsic amplification. Pin diodes may therefore be used when the generated electric output signal stands out from the noise.

In accordance with an embodiment, SiPMs and similar photodetectors are employed for monitoring the fluorescence intensity of melting DNA duplexes in DNA microarrays 15, which, for example, exhibits an amplification of 1:500,000 (a photon is transformed to a charge pulse (Q) of 500,000 electrons). SiPMs (silicon photomultipliers) consist of arrays, or arrangements, of microavalanche photodiodes that are connected in parallel and are operated in the extremely sensitive Geiger Mode.

The DNA microarray may consist of a number Y×Z of DNA samples. The DNA samples consist of one or more known DNA oligonucleotide sequences anchored at a substrate 115. In the anchored DNA, complementary oligonucleotide sequences (DNA to be examined) are hybridized. These DNA sequences have a fluorescence marker attached to them. The DNA samples in the Y×Z DNA microarray may consist of different or of identical DNA oligonucleotide sequences. The fluorescence intensity is monitored by means of an Y×Z-canalized individual photodetector or by means of a number Y×Z of separate photodetectors assembled or disposed in an array format above the DNA microarray (cf. FIG. 2). The photodetector may be based on SiPM, PIN, APD or PMT technologies. In order to be able to make use of the highly sensitive PMT technology it is useful to connect the DNA microarray and the PMT with optical waveguides since the PMT is substantially larger than the microarray. This gives rise to a bulky laboratory setting.

The thin-film heater may be patterned by means of standard MEMS manufacturing technologies and/or methods (e.g., photolithographic patterning with subsequent metallization and etching, lift-off, electroplating, metallization by means of a shadow mask) or printing methods (e.g., screen printing, inkjet, super inkjet, blade coating, gravure printing) on glass, thermoformed polymers (injection molding, hot embossing), polymer film, or silicon. The electrically conducting material of the heating resistor, or heating wire, may include a metal, a semiconductor material, conductive paste (intrinsic or doped), an electrically conductive polymer, or a conductive oxide (e.g., ITO—indium tin oxide).

FIGS. 2a and 2b further show a schematic side view of a system 30 including the measuring device 5 and the excitation light source 35 which excites the DNA sample 10 to fluoresce. The excitation light source 35 may be arranged above the DNA microarray 15, e.g., on the side of the fluorescence detector array 20, or below the DNA microarray 15, e.g., on the side of the heater 40. In the former case, the DNA sample 10 may generate fluorescence by means of reflecting light, and in the latter case, it may generate fluorescence by means of transmitted light (cf. FIGS. 3a and 3b). The integrated heating element 40 may be set up as a micro-patterned thin-film heater that is made of a grid of patterned thin-film copper, for example. The grid includes an arrangement of narrow conductor lines of a width of between 1 μm and 20 μm and having interposed transparent regions. The thickness of such a thin-film heater ranges from 100 nm to 1 μm. This enables a semi-transparent heater. In this context, the film substrate 115 is transparent. A transparent heater is advantageous for the embodiment of a direct-light fluorescence setup.

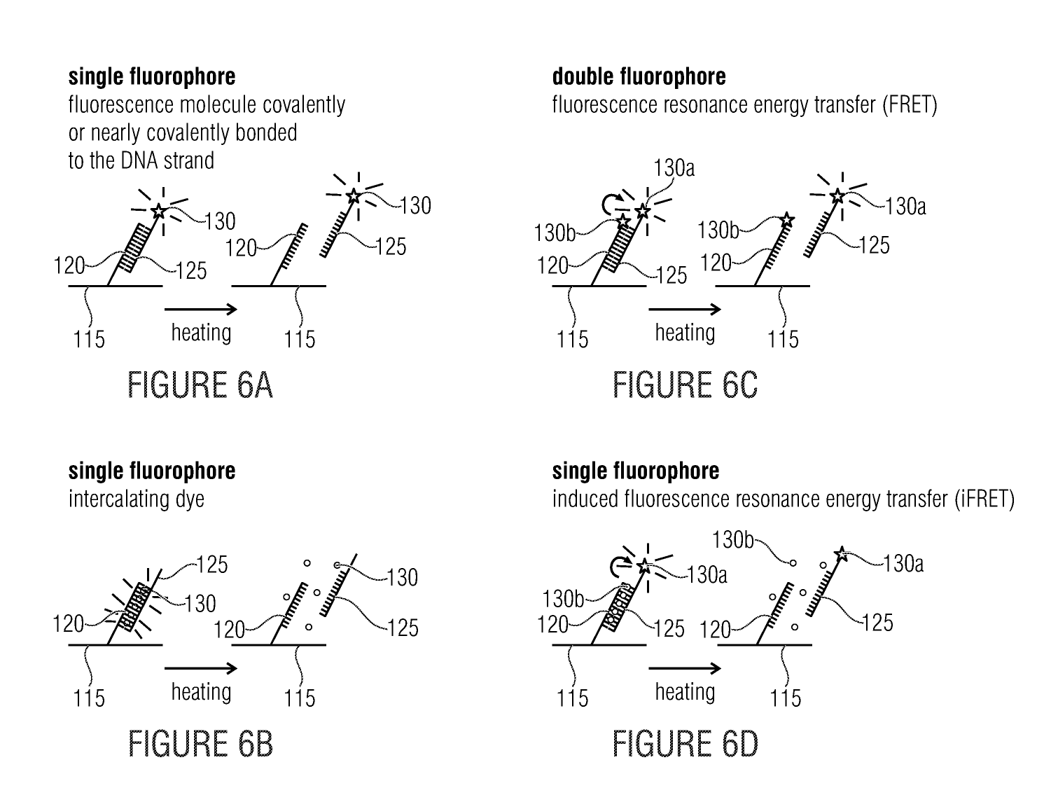

Due to being heated by the integrated heating element 40, the DNA sample 10 disassociates, i.e., the double-stranded DNA (DNA duplex) melts into single-stranded DNA. Once a DNA strand has melted, the brightness of the fluorescence decreases. The fluorescence may be created by means of intercalating dyes (cf. FIG. 6b), FRET (fluorescence resonance energy transfer) (cf. FIG. 6c), iFRET (induced fluorescence resonance transfer) (cf. FIG. 6d), or a fluorescence molecule that is covalently bonded to the target strand (cf. FIG. 6a).

Embodiments enable monitoring of the respective fluorescence intensity of each DNA spot to be performed in real time and simultaneously, or in parallel, while using a compact, portable, and low-cost detector system. The DNA microarray sample and the SiPM may be assembled either in a classic incident-light fluorescence setup (epi-fluorescence) or in a direct-light fluorescence setup (FIG. 2 and FIG. 3). Depending on the application, the setup uses additional optical components such as fluorescence filters, lenses, and dichroic mirrors, for example. The direct-light setup is advantageous since is involves fewer optical components, which is advantageous for a portable measurement setup.

FIG. 3a shows a schematic representation of a side view of the excitation light source 30, of a detector, e.g., the fluorescence detector 25, as well as of a DNA sample, e.g., the DNA sample 10. In the arrangement shown, the detector 25 is configured to detect the fluorescence of the DNA sample 10 by means of reflecting light. This classic setup is also referred to as an incident-light fluorescence setup (epi-fluorescence). A more detailed representation was already described with regard to FIG. 2a.

FIG. 3b shows a schematic representation of a side view of the measurement setup of FIG. 3a, the difference being that the excitation light source 30 and the detector 25 are arranged on different sides of the DNA sample 10. This measurement setup enables detection of the fluorescence of the DNA sample 10 by means of transmitted light. This setup is also referred to as a direct-light fluorescence setup. A more detailed representation was already described with regard to FIG. 2b.

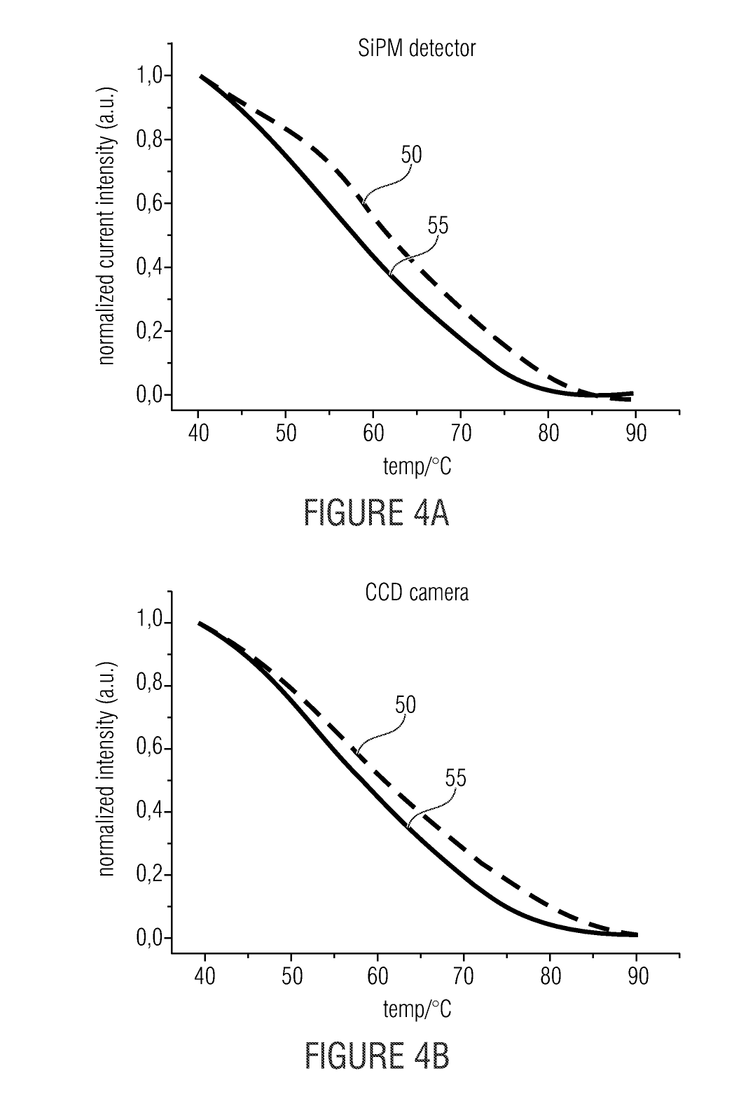

FIG. 4a shows exemplary melting curves for matched and mismatched DNA samples, i.e., samples having a mismatched base pair—SNP 50 and 55, recorded with two SiPMs and plotted as a current intensity, which is normalized to a starting value 1 and a final value 0, over the temperature. A matched DNA sample 50 is present in case same does not differ or differs only marginally from a reference sample. A mismatched DNA sample 55 may consequently comprise the deviation shown in FIG. 4a (and, subsequently, also in FIG. 4b) from the reference sample, or the matched sample. A mismatched DNA sample will occur, for example, if there is a mutation in a DNA strand. Each SiPM is directed to monitor the fluorescence intensity of an individual DNA sample.

FIG. 4b shows exemplary melting curves for the DNA samples shown in FIG. 4a, plotted as an intensity, which is normalized to a starting value 1 and a final value 0, over the temperature, which were determined by using a microscope CCD camera and by extracting the respective fluorescence intensity by means of an image software.

The exemplary melting curves, shown in FIG. 4a and FIG. 4b, of two DNA samples are immobilized in a microfluidic MCA module ([3], [4], [5]), the fluorescence intensity having been recorded while using two SiPM detectors or a conventional CCD camera and subsequent image analysis. Both measurements were performed in an incident-light mode, or incident-light fluorescence setup.

The curves in FIG. 4a and FIG. 4b exhibit a high level of similarity, which illustrates, or proves, that the two SiPMs are as good at detecting the respective fluorescence intensity of the two DNA spots as is the CCD camera. The melting curve 55, shown in FIG. 4a, of the SiPM of the mismatched DNA exhibits a deviation from the matching melting curve 50 and, thus, also from the reference curve that is even larger than the deviation, shown in FIG. 4b, of the melting curves recorded by the CCD camera.

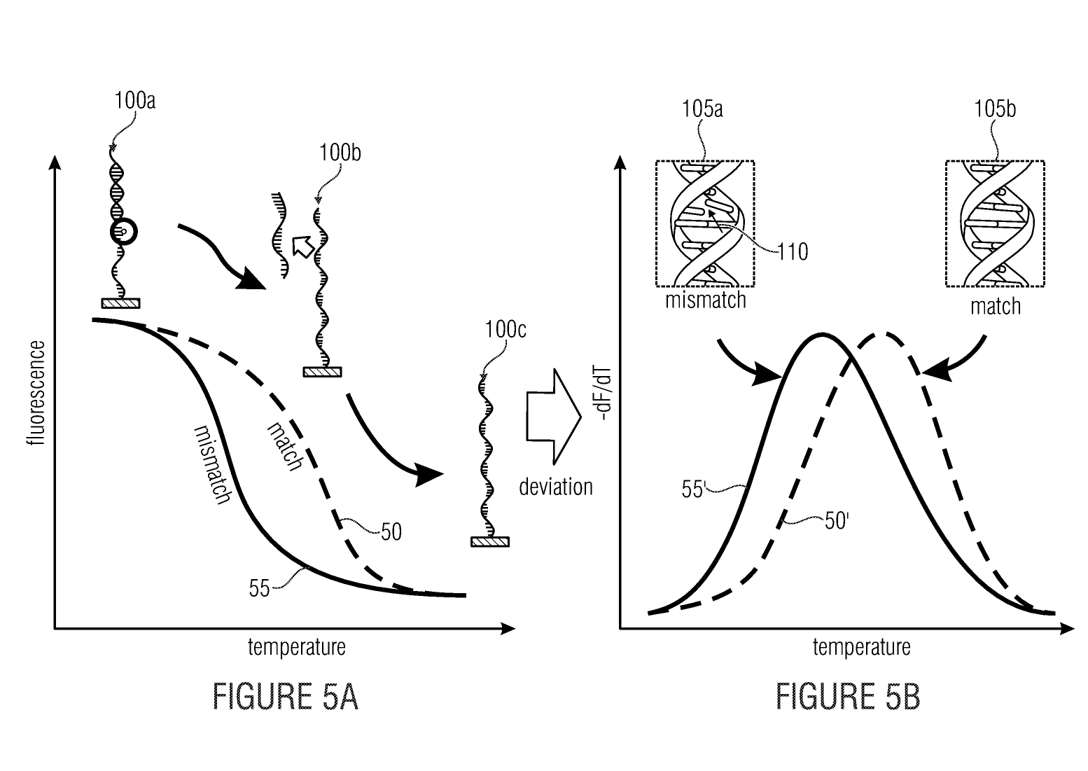

FIG. 5a shows the melting curve, which is already known from FIG. 4a and FIG. 4b, of the fluorescence over the temperature. In addition, the DNA sequences 100 exemplarily show the process taking place while the DNA is being heated. At the beginning of the heating process, double-stranded DNA 100a prevails. Due to the heating process, DNA sequences 100b which trigger the fluorescence will split off. The fluorescence decreases as the proportion of single-stranded DNA increases. This is due to various reasons which depend on the fluorescence system (cf. FIGS. 6a to 6d). In FRET and iFRET, for example, the distance between the donor molecule and the acceptor molecule is too large when the double-stranded DNA dissolves into single-stranded DNA (the complementary strand floats away). The light transfer no longer takes place and, consequently, fluorescence decreases. At the end of the heating process, single-stranded DNA sequences 100c prevail, which are no longer able to fluoresce.

FIG. 5b shows the negative derivative of the fluorescence with respect to the temperature of the melting curve of FIG. 5a; the derivative of the curve 50, which shows matched DNA sequences, results in the curve 50′. By analogy, the derivative of the curve 55 leads to the curve 55′. By analogy with FIG. 5a, DNA sequences 105 are also depicted for illustration purposes by way of example here. If there is no match for a DNA sample 105a, a bonding is missing at a location 110, as a result of which the DNA sequence can be cleaved more quickly. By contrast, the bonding within the DNA sequence 105b is complete and is thus configured to be stronger, as a result of which there is a higher level of bonding energy present and, thus, a higher temperature may be used in order to cause a change in fluorescence. This results in a lower rate of change in fluorescence.

FIG. 6 shows various fluorescence marker systems, or fluorophores 130, within the visible frequency spectrum of 400 to 800 nm by means of a known DNA oligonucleotide sequence 120 anchored on the substrate 115, as well as a complementary oligonucleotide sequence 125 hybridized at the anchored DNA. FIG. 6a and FIG. 6b show individual fluorophores 130 which, in FIG. 6a, enter into a covalent or nearly covalent bonding with the DNA strand 125 by means of a conventional dye, or quantum dot (e.g., Cy3). FIG. 6b shows an intercalating dye 130 in the form of a dye which is specific to double-stranded DNA and exhibits a higher binding affinity with double-stranded DNA than with single-stranded DNA and which changes its intensity or emission wavelength in the absence of double-stranded DNA. Thus, the fluorescence decreases when the DNA sequences 120 and 125 are cleaved.

FIGS. 6c and 6d show double fluorophores 130a and 130b. FIG. 6c shows a FRET (fluorescence resonance energy transfer). Here, an emission light 130b of the fluorophore present on an immobilized DNA strand 120 (donor) is made use of in order to excite the fluorophore 130a present on the complementary DNA strand (acceptor). The donor and acceptor fluorophores are covalently bonded on the known DNA 120 and the DNA strand 125 to be examined. If the bonding between the two DNA strands 120 and 125 is dissolved, the fluorophore 130a present on the DNA strand 125 to be examined will no longer be excited by the fluorophore 130b and will thus reduce its fluorescence. FIG. 6d shows an iFRET (induced fluorescence resonance energy transfer) fluorophore which makes use of emitted light from an intercalating dye 130b so as to excite immobilized fluorophores 130a present on complementary DNA strands. Here, the fluorescence also decreases when the DNA strand 125 to be examined detaches from the known DNA strand 120.

In general terms, the described embodiments relate to a measuring device for simultaneously analyzing melting curves of DNA samples in DNA microarrays with the aid of a fluorescence detector array. Embodiments show monitoring of melting curves of DNA microarrays (dynamic fluorescence measurement) by means of silicon photomultipliers (SiPM) or other photodetectors such as, e.g., PIN diodes (positive intrinsic negative diodes), avalanche photodiodes (APD), or photomultiplier tubes (PMT). The DNA microarray is applied, along with a thin-film heating element, to a substrate made of plastic or glass or is integrated in a microfluidic channel. However, the scope of protection will be defined by the claims which follow.

According to embodiments, the fluorescence detector array 20 may comprise a CCD (charge coupled device) or a CMOS (complementary metal oxide semiconductor) detector or sensor. Therefore, single CCD or CMOS detectors may be formed into the fluorescence detector array 20, wherein the single CCD or CMOS detector forms one fluorescence detector. Further embodiments show that one or more CCD or CMOS detectors may be used as a fluorescence detector array, where a single pixel or a group of pixels is used as a single fluorescence detector or as a channel of a monolithic multichannel fluorescence detector. Furthermore, an embodiment shows the CCD or CMOS detector using an image analysis system to extract the fluorescence of each pixel of the detector. Moreover, the image analysis system may perform or implement a software-based approach to perform the relation of the single pixel or the group of pixels to the DNA sample. This may be performed e.g. by calculating an angle of the incident or incoming fluorescence radiation at each pixel of the CCD or CMOS detector to separate the total amount of incident fluorescence radiation into a discrete set of angles, where each angle relates to a single DNA sample.

Even though some aspects were described in connection with a device, it shall be understood that said aspects also represent a description of the corresponding method, so that a block or a structural component of a device is also to be understood as a corresponding method step or as a feature of a method step. By analogy therewith, aspects that were described in connection with or as a method step also represent a description of a corresponding block or detail or feature of a corresponding device.

While this invention has been described in terms of several embodiments, there are alterations, permutations, and equivalents which fall within the scope of this invention. It should also be noted that there are many alternative ways of implementing the methods and compositions of the present invention. It is therefore intended that the following appended claims be interpreted as including all such alterations, permutations and equivalents as fall within the true spirit and scope of the present invention.

### SOURCES

[1] “DASH-2: Flexible, Low-Cost, and High-Throughput SNP Genotyping by Dynamic Allele-Specific Hybridization on Membrane Arrays”; M. Jobs, W M Howell, L. Stromqvist, T. Mayr, A J Brookes. Genome Res. 2003 May; 13(5):916-24
[2] “DNA Diagnostics by Surface-Bound Melt-Curve Reactions” L. Strömqvist Meuzelaar, K. Hopkins, E. Liebana, A. J. Brookes; Journal of Molecular Diagnostic, Vol. 9, No. 1, February 2007.
[3] “DNA melting curve analysis on semi-transparent thin film microheater on flexible lab-on-foil substrate” A. Ohlander, T. Hammerle, G. Klink, C. Zilio, F. Damin, M. Chiari, A. Russom, K. Brock. μTAS 2012, M.8.180.
[4] “Genotyping of single nucleotide polymorphisms by melting curve analysis using thin film semi-transparent heaters in a lab-on-foil system”A. Ohlander, C. Zilio, T. Hammerle, S. Zelenin, G. Klink, M. Chiari, K. Bock, A. Russom,: Lab Chip. 2013 Jun. 7; 13(11):2075-82.
[5] “Foil-based DNA melting curve analysis platform for low-cost point of care molecular diagnostics”: A. Ohlander, S. Bauer, H. Ramachandraiah, A. Russom, K. Bock; microTAS 2013, Freiburg, Germany.
[6] A. Ohlander, G. Klink, A. Russom, K. Bock. “Semitransparent heater for biological and chemical assays” DE 102012219656.

1. A measuring device for performing melting curve analysis of at least one DNA sample, the measuring device comprising:
a two-dimensional DNA microarray for accommodating the at least one DNA sample;
a two-dimensional fluorescence detector array comprising at least one fluorescence detector; and
an integrated heating element for heating the DNA sample applied to the DNA microarray.

2. The measuring device as claimed in claim 1, wherein the fluorescence detector comprises a silicon photomultiplier.

3. The measuring device as claimed in claim 1, wherein the fluorescence detector comprises an avalanche photodiode.

4. The measuring device as claimed in claim 1, wherein the fluorescence detector comprises a PIN diode.

5. The measuring device as claimed in claim 1, wherein the fluorescence detector comprises a photomultiplier tube and an optical waveguide.

6. The measuring device as claimed in claim 1, wherein the fluorescence detector is configured to detect a change in the fluorescence intensity of the at least one DNA sample that is caused by a change in a nucleic-acid structure that is due to a temperature rise.

7. The measuring device as claimed in claim 1, wherein the fluorescence detector array comprises a plurality of fluorescence detectors or a monolithic multichannel fluorescence detector.

8. The measuring device as claimed in claim 7, wherein each of the plurality of fluorescence detectors or each channel of the multichannel fluorescence detector is coupled to a DNA sample by means of an optical waveguide or imaging optics.

9. The measuring device as claimed in claim 1, wherein the measuring device is configured to simultaneously analyze a plurality of DNA samples on the DNA microarray.

10. The measuring device as claimed in claim 9, wherein each of the plurality of DNA samples is associated with a fluorescence detector of the fluorescence detector array.

11. The measuring device as claimed in claim 1, the measuring device being portable.

12. The measuring device as claimed in claim 1, wherein excitation of the fluorescence is performed using a direct-light method with a transparent substrate and a transparent or semi-transparent integrated heating element.

13. The measuring device as claimed in claim 1, wherein excitation of the fluorescence is performed using an incident-light method.

14. The measuring device as claimed in claim 1, wherein the fluorescence detector is configured to react to an input light intensity, which is due to a fluorescence of the at least one DNA sample, with an amplified output signal.

15. A system comprising:
a measuring device as claimed in claim 1; and
an excitation light source which excites the at least one DNA sample to fluoresce.

16. Utilization of a fluorescence detector array for performing melting curve analysis of a DNA sample, at least one fluorescence detector of the fluorescence detector array being a silicon photomultiplier (SiPM).

17. A measuring device for performing melting curve analysis of Y×Z DNA samples, the measuring device comprising:
a DNA microarray for accommodating the Y DNA samples arranged along a Y extension and the Z DNA samples arranged along a Z extension;
a fluorescence detector array comprising Y×Z separate fluorescence detectors, Y fluorescence detectors being arranged along a Y extension and Z fluorescence detectors being arranged along a Z extension, or a monolithic Y×Z multichannel fluorescence detector, Y channels of the multichannel fluorescence detector being arranged along a Y extension and Z channels of the multichannel fluorescence detector being arranged along a Z extension; and
an integrated heating element for heating the DNA samples applied to the DNA microarray;
the measuring device being configured to analyse the Y×Z DNA samples on the DNA microarray at the same time; and
each of the Y×Z DNA samples being aligned to be spatially opposite one of the Y×Z fluorescence detectors of the fluorescence detector array or one of the Y×Z channels des multichannel fluorescence detector and thus being associated with a corresponding fluorescence detector or channel.

18. Utilization of a fluorescence detector array for performing melting curve analysis of a DNA sample, at least one fluorescence detector of the fluorescence detector array comprising a group of individual silicon photomultipliers (SiPM).
